(19) United States
(12) Patent Application Publication
(10) Pub. No.: US 2023/0157845 A1
(43) Pub. Date: May 25, 2023

(54) FEMORAL NECK MEASURING METHOD AND SYSTEM BASED ON OPTICAL LOCALIZATION, DEVICE AND MEDIUM.

(71) Applicants: Shantou University, Shantou (CN); The First Affiliated Hospital of Shantou University Medical College, Shantou (CN)

(72) Inventors: Zhun Fan, Shantou (CN); Weibo Ning, Shantou (CN); Jiazi Liu, Shantou (CN); Jiaqi Zhu, Shantou (CN); Guijie Zhu, Shantou (CN); Jun Hu, Shantou (CN); Hongjiang Chen, Shantou (CN); Jiacheng Liu, Shantou (CN); Zhifeng Hao, Shantou (CN)

(73) Assignees: Shantou University, Shantou (CN); The First Affiliated Hospital of Shantou University Medical College, Shantou (CN)

(21) Appl. No.: 18/049,681

(22) Filed: Oct. 26, 2022

(30) Foreign Application Priority Data
Nov. 22, 2021 (CN) 202111391356.9

(51) Int. Cl. A61F 2/46; A61B 5/00

(52) U.S. Cl. CPC A61F 2/4657; A61F 2/4607; A61B 5/4887; A61F 2002/4658; A61F 2002/4633

(57) ABSTRACT

A femoral neck measuring method includes: measuring an affected limb after a femoral stem prosthesis is installed through an optical localization probe, and calculating an actual length of the femoral neck needed by the affected limb according to an osteotomy surface of the affected limb determined after the femoral stem prosthesis is installed; determining a femoral ball prosthesis and a femoral neck prosthesis to be selected according to the calculated actual length of the femoral neck and a length of a femoral neck of a healthy limb measured before surgery; and measuring a length of the affected limb after the femoral ball prosthesis and the femoral neck prosthesis are implanted, and restraining a length difference between the healthy limb and the affected limb by adjusting a length of the femoral neck of the affected limb.

### CROSS-REFERENCE TO RELATED APPLICATION

This application is based on and claims the benefit of priority from Chinese Patent Application No. 2021113913569, filed on 22 Nov. 2021, the entirety of which is incorporated by reference herein.

### TECHNICAL FIELD

The disclosure relates to the technical field of medical treatment, in particular to a femoral neck measuring method and system based on optical localization, a device and a medium.

### BACKGROUND

The elderly are at high risk of hip joint diseases due to the declined bone qualities of the elderly. With the development of the aging society, total hip arthroplasty (THA) is one of the commonly used methods to treat related hip joint diseases. However, the femur used in the total hip arthroplasty depends on planning before surgery, and coping strategies of adjusting a length of the femoral neck during surgery are scarce.

In the existing technology, there are two methods for adjusting the length of the femoral neck during the total hip arthroplasty: (1) a distance between two reference points is measured by using a vertical ruler, a vernier caliper, a kirschner wire, or the like, to determine femoral eccentricity, so as to adjust the length of the femoral neck. The difficulty of operation is low, but it is necessary to directly interfere with an affected part of a patient for surgery, which may interfere with the surgery of doctors, and the universality is low, which is not convenient for the doctors to read quickly and visually; (2) a computer-aided imaging system is used to measure morphological parameters of the femoral neck. Accurate measurement can be ensured, but the process is complicated and the operation is difficult.

### SUMMARY

The object of the disclosure is to provide a femoral neck measuring method and system based on optical localization, a device and a medium, so as to solve one or more technical problems in the existing technology, and at least provides a beneficial choice or creation condition.

According to a first aspect, a femoral neck measuring method based on optical localization is provided, including:

measuring an affected limb after a femoral stem prosthesis is installed through an optical localization probe, and calculating an actual length of the femoral neck needed by the affected limb according to an osteotomy surface of the affected limb determined after the femoral stem prosthesis is installed;

determining a femoral ball prosthesis and a femoral neck prosthesis to be selected according to the calculated actual length of the femoral neck and a length of a femoral neck of a healthy limb measured before surgery; and

measuring a length of the affected limb after the femoral ball prosthesis and the femoral neck prosthesis are implanted, and restraining a length difference between the healthy limb and the affected limb by adjusting a length of the femoral neck of the affected limb.

In some embodiments, measuring an affected limb after a femoral stem prosthesis is installed through an optical localization probe, and calculating an actual length of the femoral neck needed by the affected limb according to an osteotomy surface of the affected limb determined after the femoral stem prosthesis is installed, include:

measuring three non-collinear reference points on the osteotomy surface of the affected limb by using the optical localization probe, and determining coordinates of the three reference points;

pre-installing the femoral ball prosthesis and the femoral neck prosthesis selected before surgery, measuring a measuring point of the femoral ball prosthesis far away from the osteotomy surface by using the optical localization probe, and determining coordinates of the measuring point of the femoral ball prosthesis; and

determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis.

In some embodiments, determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis, include:

substituting coordinate variables of the three reference points into a plane formula of the osteotomy surface of the affected limb for solution;

the plane formula of the osteotomy surface of the affected limb being:

ax+by+cz+d=0;

wherein, a, b, c and d are all constants, while x, y and z respectively represent the coordinate values of the points on the osteotomy surface of the affected limb;

calculating the distance between the osteotomy surface of the affected limb and the measuring point by using a distance calculation formula;

the distance calculation formula being:

$$e = \frac{{ax}_{4} + {by}_{4} + {cz}_{4} + d}{\sqrt{{a}^{2} + {b}^{2} + {c}^{2}}} ;$$

wherein, x₄, y₄ and z₄ respectively represent the three coordinate values of the measuring point, and e represents the distance between the osteotomy surface of the affected limb and the measuring point; and

subtracting a radius length of the pre-selected femoral ball prosthesis from the distance between the osteotomy surface of the affected limb and the measuring point to obtain the actual length of the femoral neck.

In some embodiments, determining a femoral ball prosthesis and a femoral neck prosthesis to be selected according to the calculated actual length of the femoral neck and a length of a femoral neck of a healthy limb measured before surgery includes:

selecting a femoral neck prosthesis again according to the actual length of the femoral neck and the length of the femoral neck of the healthy limb; and

determining a size of the femoral ball prosthesis according to a size of the femoral neck prosthesis selected and the length of the femoral neck of the healthy limb, and selecting a final femoral ball prosthesis.

In some embodiments, selecting a final femoral ball prosthesis includes:

inputting the size of the femoral neck prosthesis selected into a preset femoral ball prosthesis database, and selecting the femoral ball prosthesis from femoral ball prosthesis models output from the femoral ball prosthesis database.

In some embodiments, measuring a length of the affected limb after the femoral ball prosthesis and the femoral neck prosthesis are implanted, and restraining a length difference between the healthy limb and the affected limb by adjusting a length of the femoral neck of the affected limb, include:

after implanting the femoral ball prosthesis and the femoral neck prosthesis into the affected limb, measuring a length between an anterior superior iliac spine and a pelma of the affected limb to obtain the length of the affected limb;

comparing the length of the affected limb with the length of the healthy limb measured before surgery, and determining the length difference between the healthy limb and the affected limb; and

judging whether the length difference between the affected limb and the healthy limb exceeds a set value for unequal lengths of both lower limbs, and in response to the length difference exceeding the set value, replacing the femoral ball prosthesis and/or the femoral neck prosthesis so that the difference between the length of the affected limb and the length of the healthy limb is within the set value for unequal lengths of both lower limbs.

In some embodiments, the set value for unequal lengths of both lower limbs is 10 mm.

According to a second aspect, a femoral neck measuring system based on optical localization is provided, including:

a detection module configured for measuring an affected limb after a femoral stem prosthesis is installed through an optical localization probe, and calculating an actual length of the femoral neck needed by the affected limb according to an osteotomy surface of the affected limb determined after the femoral stem prosthesis is installed;

a first adjustment module configured for determining a femoral ball prosthesis and a femoral neck prosthesis to be selected according to the calculated actual length of the femoral neck and a length of a femoral neck of a healthy limb measured before surgery; and

a second adjustment module configured for measuring a length of the affected limb after the femoral ball prosthesis and the femoral neck prosthesis are implanted, and restraining a length difference between the healthy limb and the affected limb by adjusting a length of the femoral neck of the affected limb.

According to a third aspect, a computer device is provided, including:

a memory storing a computer program thereon; and

a processor which, when executing the computer program, implements the femoral neck measuring method based on optical localization according to the first aspect.

According to a fourth aspect, a non-transitory computer readable storage medium storing a computer program thereon is provided, wherein the computer program, when executed by a processor, implements the femoral neck measuring method based on optical localization according to the first aspect.

The disclosure has the beneficial effects as follows. Errors caused by surgical operation are measured and calculated by using an optical measurement technology in total hip arthroplasty, so that an actual effect achieved in the surgical execution process can be determined while the operation is relatively simple, and medical staff can adjust the selected prosthesis in time according to each stage of the surgery to improve the surgery quality.

### BRIEF DESCRIPTION OF THE DRAWINGS

FIG. 1 is a flow chart of a femoral neck measuring method based on optical localization according to an embodiment of the disclosure.

FIG. 2 is a schematic structural diagram of implanting a prosthesis into an affected limb in total hip arthroplasty.

FIG. 3 is a flow chart of the method of step S100 according to an embodiment of the disclosure.

FIG. 4 is a flow chart of the method of step S200 according to an embodiment of the disclosure.

FIG. 5 is a flow chart of the method of step S300 according to an embodiment of the disclosure.

FIG. 6 is a structural block diagram of a femoral neck measuring system based on optical localization according to an embodiment of the disclosure.

FIG. 7 is an internal structure diagram of a computer device according to an embodiment of the disclosure.

### DETAILED DESCRIPTION

In order to make the objects, technical solutions and advantages of the embodiments of the disclosure clearer, the disclosure will be further described below with reference to the embodiments and drawings.

Reference will now be made in detail to exemplary embodiments, examples of which are illustrated in the drawings. When the following description refers to the drawings, unless otherwise indicated, the same numbers in different drawings indicate the same or similar elements. The implementations set forth in the following description of exemplary embodiments do not represent all the implementations consistent with the present disclosure. On the contrary, they are only examples of devices and methods consistent with some aspects of the present disclosure as detailed in the appended claims.

According to a first aspect of the disclosure, a femoral neck measuring method based on optical localization is provided.

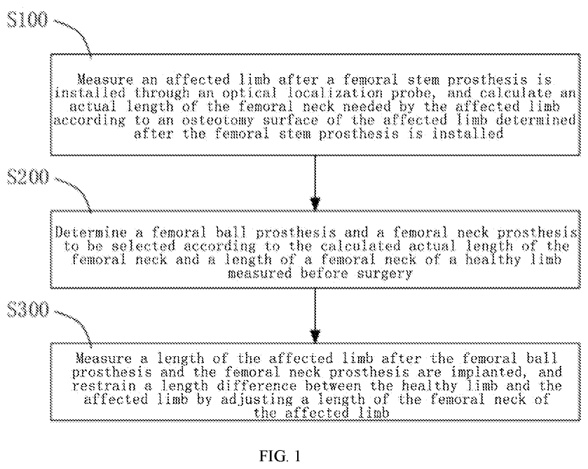

Referring to FIG. 1, FIG. 1 is a flow chart of a femoral neck measuring method based on optical localization according to an embodiment of the disclosure. As shown in FIG. 1, the method includes the following step S100 to step S300.

At step S100, an affected limb after a femoral stem prosthesis is installed is measured through an optical localization probe, and an actual length of the femoral neck needed by the affected limb is calculated according to an osteotomy surface of the affected limb determined after the femoral stem prosthesis is installed.

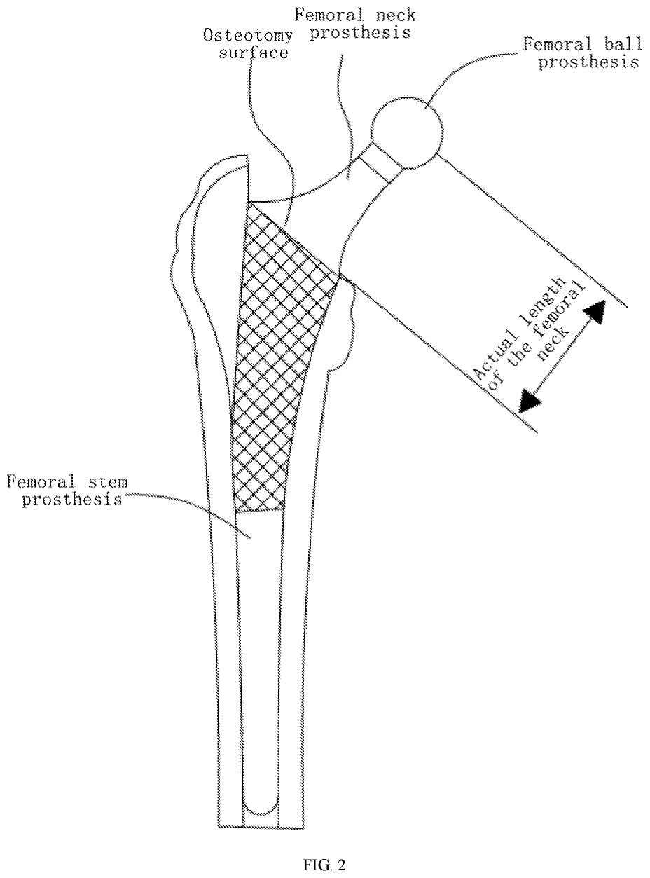

As shown in FIG. 2, the prosthesis implanted into the affected limb in total hip replacement includes a femoral stem prosthesis, a femoral neck prosthesis and a femoral ball prosthesis. In the existing technology, after all the prostheses are installed according to parameters measured and designed before surgery, the difference between two lower limbs is judged by doctors according to their experiences, but the knowledge of the difference between the two lower limbs caused by a slight error of the implantation depth of the femoral stem prosthesis in surgery is lacking.

At step S100, detection is carried out after the femoral stem prosthesis is implanted into the affected limb. After the femoral stem prosthesis is implanted into the affected limb, a slope of an upper surface of the femoral stem prosthesis is used as a femoral surface of the affected limb. After the femoral neck prosthesis and the femoral ball prosthesis pre-selected before surgery are implanted into the femoral surface formed by the femoral stem prosthesis, the actual length of the femoral neck needed by the affected limb is measured by the optical localization probe and calculated in an optical localization system, so that medical staff can know the actual effect produced by the pre-selected prosthesis combination when the implanting operation is executed during the surgery.

One or more balls are fixed on the optical localization probe. A measuring principle of the optical localization probe is to track the optical localization balls from different angles by using optical sensors, and then determine three-dimensional coordinate positions of the optical localization balls in space. When the optical localization probe attached with the optical localization balls is placed under the optical localization system, each optical localization ball is used to locate a probe tip, so as to track a tail end of the optical localization probe and determine three-dimensional coordinates of the tail end of the optical localization probe, and then determine three-dimensional coordinates of a point to be measured.

At step S200, a femoral ball prosthesis and a femoral neck prosthesis to be selected are determined according to the calculated actual length of the femoral neck and a length of a femoral neck of a healthy limb measured before surgery.

Comparing the actual length of the femoral neck calculated above with the length of the femoral neck of the healthy limb measured by CT before surgery, the medical staff can quickly determine whether the prosthesis combination pre-selected before surgery can achieve the installation effect that the lengths of both limbs of the patient are the same. If an overall error caused by the implanting error of the femoral stem prosthesis is within an acceptable range, the femoral ball prosthesis and the femoral neck prosthesis pre-selected before surgery can be maintained, otherwise, femoral ball prosthesis and femoral neck prosthesis of different models or sizes need to be selected again.

At step S300, a length of the affected limb after the femoral ball prosthesis and the femoral neck prosthesis are implanted is measured, and a length difference between the healthy limb and the affected limb is restrained by adjusting a length of the femoral neck of the affected limb.

After correcting the errors in the surgery process through the above steps, at step S300, secondary detection is carried out on the affected limb which has initially completed prosthesis implantation, the length of the affected limb is compared with that of the healthy limb, and the length difference between the healthy limb and the affected limb is restrained within the reasonable error range to improve the surgery quality. The core idea of restraining the length difference between the healthy limb and the affected limb is to adjust the length of the femoral neck of the affected limb, which can be realized by selecting the femoral ball prosthesis and/or the femoral neck prosthesis again.

In this way, errors caused by surgical operation are measured and calculated by using an optical measurement technology in total hip arthroplasty, so that an actual effect achieved in the surgical execution process can be determined while the operation is relatively simple, and medical staff can adjust the selected prosthesis in time according to each stage of the surgery to improve the surgery quality.

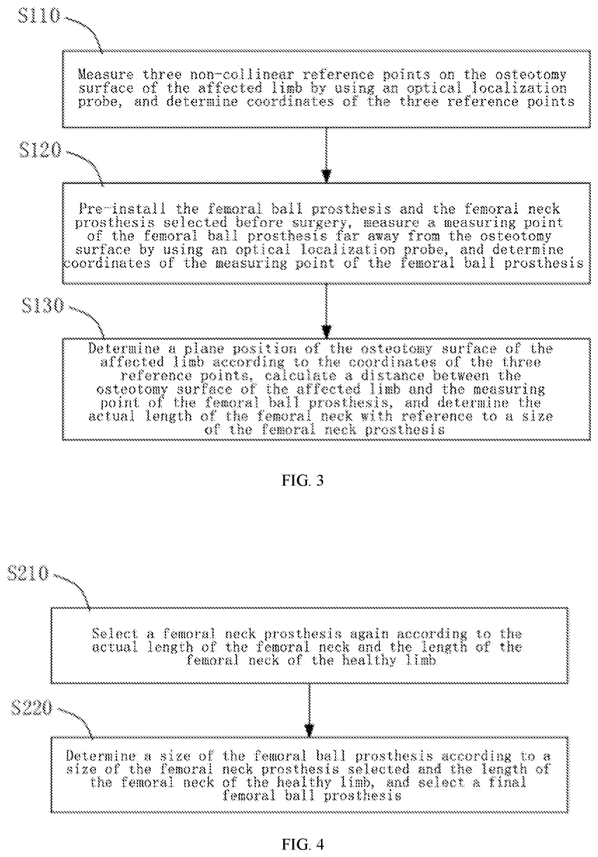

Referring to FIG. 3, FIG. 3 is a flow chart of the method of step S100 according to an embodiment of the disclosure. As shown in FIG. 3, the method includes the following step S110 to step S130.

At step S110, three non-collinear reference points on the osteotomy surface of the affected limb are measured by using the optical localization probe, and coordinates of the three reference points are determined.

At step S120, the femoral ball prosthesis and the femoral neck prosthesis selected before surgery are pre-stalled, a measuring point of the femoral ball prosthesis far away from the osteotomy surface is measured by using an optical localization probe, and coordinates of the measuring point of the femoral ball prosthesis are determined.

At step S130, a plane position of the osteotomy surface of the affected limb is determined according to the coordinates of the three reference points, a distance between the osteotomy surface of the affected limb and the measuring point of the femoral ball prosthesis is calculated, and the actual length of the femoral neck is determined with reference to a size of the femoral neck prosthesis.

This embodiment provides a method for measuring and calculating the actual length of the femoral neck.

At step S110, the coordinates of the three reference points are measured by using the optical positioning probe and respectively represented as (x₁, y₁, z₁), (x₂, y₂, z₂) and (x₃, y₃, z₃).

At step S120, after installing the pre-selected femoral ball prosthesis and the pre-selected femoral neck prosthesis on the osteotomy surface of the femoral stem prosthesis, as shown in FIG. 2, the point in an area far away from the femoral neck prosthesis is selected as the measuring point of the femoral ball prosthesis, and the measuring point is a point on the femoral ball prosthesis farthest from the femoral neck prosthesis.

At step S130, coordinate variables of the three reference points are substituted into a plane formula of the osteotomy surface of the affected limb for solution.

After the coordinates (x₁, y₁, z₁), (x₂, y₂, z₂) and (x₃, y₃, z₃) of the three reference points are substituted into the plane formula of the osteotomy surface of the affected limb, expression of the osteotomy surface of the affected limb in the optical localization system is obtained, and the plane formula of the osteotomy surface of the affected limb is as follows:

ax+by+cz+d=0;

wherein, a, b, c and d are all constants, while x, y and z respectively represent the coordinate values of the points on the osteotomy surface of the affected limb.

The distance between the osteotomy surface of the affected limb and the measuring point is calculated by using a distance calculation formula.

The distance calculation formula is:

$$e = \frac{{ax}_{4} + {by}_{4} + {cz}_{4} + d}{\sqrt{{a}^{2} + {b}^{2} + {c}^{2}}} ;$$

wherein, x₄, y₄ and z₄ respectively represent the three coordinate values of the measuring point, and e represents the distance between the osteotomy surface of the affected limb and the measuring point.

A radius length of the pre-selected femoral ball prosthesis is subtracted from the distance between the osteotomy surface of the affected limb and the measuring point to obtain the actual length of the femoral neck.

Referring to FIG. 4, FIG. 4 is a flow chart of the method of step S200 according to an embodiment of the disclosure. As shown in FIG. 4, the method includes the following step S210 to step S220.

At step S210, a femoral neck prosthesis is selected again according to the actual length of the femoral neck and the length of the femoral neck of the healthy limb.

At step S220, the size of the femoral ball prosthesis is determined according to a size of the femoral neck prosthesis selected and the length of the femoral neck of the healthy limb, and the final femoral ball prosthesis is selected.

In this embodiment, when the length difference between the actual length of the femoral neck and the length of the femoral neck of the healthy limb is too large, an appropriate femoral neck prosthesis is selected again, so that the length of the femoral neck prosthesis of the affected limb is approximately the same as the length of the femoral neck of the healthy limb. On this basis, the finally selected femoral ball prosthesis is determined again, so that the length of the affected limb after installing the prosthesis is the same as that of the healthy limb at the same position.

When selecting the femoral ball prosthesis, because the femoral ball prosthesis has different models, connection depth and radius between the femoral ball prosthesis of each model and the femoral neck prosthesis are different, which may lead to the deviation of the length of the affected limb. In this embodiment, the preset femoral ball prosthesis database is used to record parameters of each model of femoral ball prosthesis, the size of the femoral neck prosthesis selected is input into the preset femoral ball prosthesis database, and the femoral ball prosthesis is selected from the femoral ball prosthesis models output from the femoral ball prosthesis database.

Referring to FIG. 5, FIG. 5 is a flow chart of the method of step S300 according to an embodiment of the disclosure. As shown in FIG. 5, the method includes the following step S310 to step S330.

At step S310, after implanting the femoral ball prosthesis and the femoral neck prosthesis into the affected limb, a length between an anterior superior iliac spine and a pelma of the affected limb is measured to obtain the length of the affected limb.

At step S320, the length of the affected limb is compared with the length of the healthy limb measured before surgery, and the length difference between the healthy limb and the affected limb is determined.

At step S330, it is judged whether the length difference between the affected limb and the healthy limb exceeds a set value for unequal lengths of both lower limbs, and when the length difference exceeds the set value, the femoral ball prosthesis and/or the femoral neck prosthesis is replaced so that the difference between the length of the affected limb and the length of the healthy limb is within the set value for unequal lengths of both lower limbs.

With the aid of the image-free surgical navigation system based on the principle of optical localization, after the prosthesis is installed according to the measurement parameters, the medical staff can use the probe to measure the lengths of both lower limbs of the patient in real time, and make adjustments according to real-time feedback information, determine the lengths of the affected limb and the healthy limb by measuring the length between the anterior superior iliac spine and the pelma, so as to determine whether the length difference between both lower limbs exceeds the set value for unequal lengths of both lower limbs.

The set value for unequal lengths of both lower limbs is 10 mm, and it is clinically defined that both lower limbs are unequal in length when the difference between both lower limbs is greater than 10 mm; and if the corresponding difference between the lengths of both lower limbs is greater than 10 mm, the length of the femoral neck of the prosthesis needs to be adjusted until the error is within an allowable range.

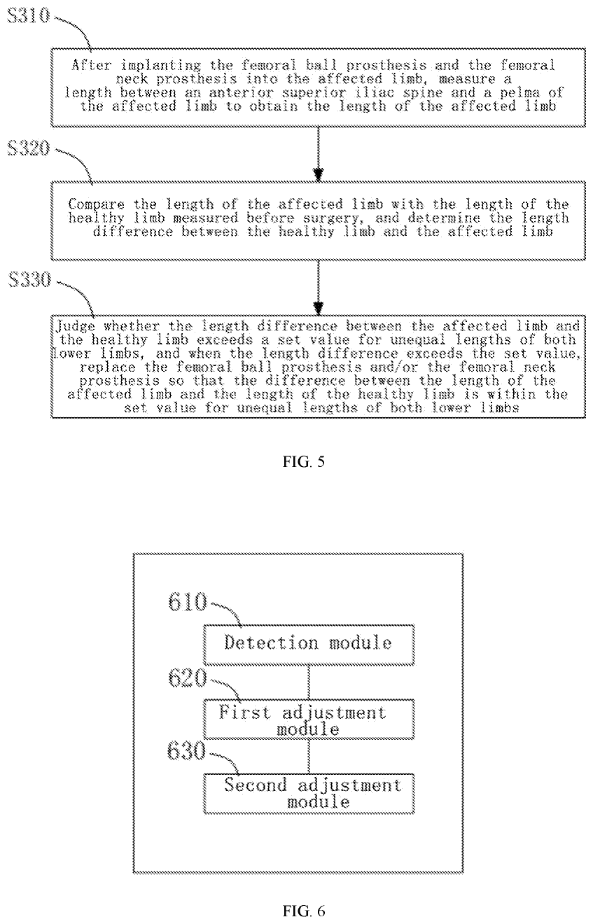

Referring to FIG. 6, FIG. 6 is a structural block diagram of a femoral neck measuring system based on optical localization according to an embodiment of the disclosure. As shown in FIG. 6, the system includes:

a detection module 610 configured for measuring an affected limb after a femoral stem prosthesis is installed through an optical localization probe, and calculating an actual length of the femoral neck needed by the affected limb according to an osteotomy surface of the affected limb determined after the femoral stem prosthesis is installed;

a first adjustment module 620 configured for determining a femoral ball prosthesis and a femoral neck prosthesis to be selected according to the calculated actual length of the femoral neck and a length of a femoral neck of a healthy limb measured before surgery; and

a second adjustment module 630 configured for measuring a length of the affected limb after the femoral ball prosthesis and the femoral neck prosthesis are implanted, and restraining a length difference between the healthy limb and the affected limb by adjusting a length of the femoral neck of the affected limb.

The femoral neck measuring system based on optical localization executes the femoral neck measuring method based on optical localization according to the first aspect. For the specific definition of the femoral neck measuring system based on optical localization, please refer to the above definition of the femoral neck measuring method based on optical localization, which will not be elaborated herein.

Each module in the above-mentioned femoral neck measuring system based on optical localization may be implemented completely or partially by software, hardware and combinations thereof The above modules may be embedded in or independent of a processor in a computer device in the form of hardware, or stored in a memory in the computer device in the form of software, so that the processor can call and execute the operations corresponding to the above modules.

According to a third aspect of the disclosure, a computer device is provided.

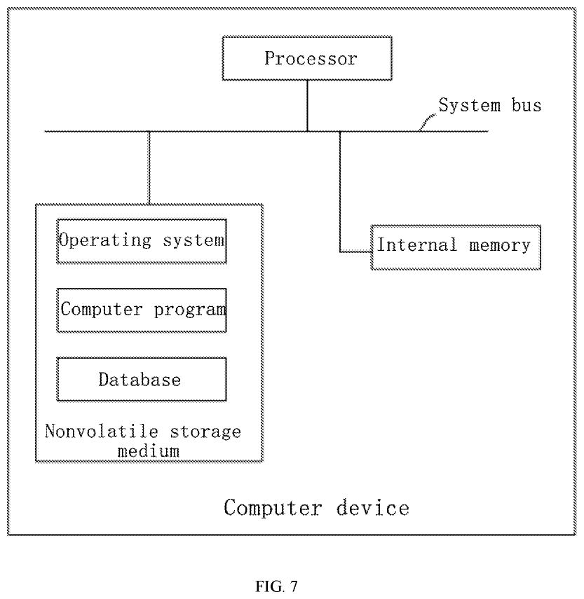

Referring to FIG. 7, FIG. 7 is an internal structure diagram of a computer device according to an embodiment of the disclosure. As shown in FIG. 7, the computer device includes a processor, a memory and a database connected via a system bus. The processor of the computer device is configured for providing calculation and control capacities. The memory of the computer device includes a nonvolatile storage medium and an internal memory. The nonvolatile storage medium stores an operating system, a computer program and a database. The internal memory provides an environment for the running of the operating system and the computer program in the nonvolatile storage medium. The computer program, when being executed by the processor, implements the femoral neck measuring method based on optical localization according to the first aspect.

All elements of the memory and the processor are directly or indirectly electrically connected with each other to realize data transmission or interaction. For example, these elements may be electrically connected with each other through one or more communication buses or signal lines. The processor is configured for processing measurement data and outputting calculation results, and the processor includes at least one software function module which may be stored in the memory in the form of software or firmware or solidified in an operating system (OS) of a server. The processor is configured for executing an executable module stored in the memory.

The memory may be Random Access Memory (RAM), Read Only Memory (ROM), Programmable Read-Only Memory (PROM), Erasable Programmable Read-Only Memory (EPROM), Electric Erasable Programmable Read-Only Memory (EEPROM), and the like. The memory is configured for storing programs and voice data, and the processor executes the programs after receiving execution instructions.

The processor may be an integrated circuit chip with a signal processing capacity. The above-mentioned processor may be a general-purpose processor, including Central Processing Unit (referred to as CPU), Network Processor (referred to as NP); and may also be Digital Signal Processor (DSP), Application Specific Integrated Circuit (ASIC), Field Programmable Gate Array (FPGA) or other programmable logic devices, discrete gate or transistor logic devices, discrete hardware components. The methods, steps, and logic diagrams in the embodiments of the disclosure may be implemented or executed by the processor. The general-purpose processor may be a microprocessor or the processor may be any conventional processor, or the like.

The processor couples various input/output devices to the processor and the memory. In some embodiments, the processor and the memory may be implemented in a single chip. In some other examples, they may be implemented by independent chips.

A peripheral interface couples various input/output devices to the processor and the memory. In some embodiments, the peripheral interface, the processor and the memory may be implemented in a single chip. In some other examples, they may be implemented by independent chips.

According to a fourth aspect of the disclosure, a computer storage medium is further provided. A computer program is stored in the computer storage medium, and the computer storage medium may be a magnetic random access memory, a read-only memory, a programmable read-only memory, an erasable programmable read-only memory, an electrical erasable programmable read-only memory, a flash memory, a magnetic surface memory, an optical disk, or a read-only optical disk, and the like; and may also be various devices including one or any combination of the above memories, such as a mobile phone, a computer, a tablet device, a personal digital assistant, and the like. This computer program, when being executed by the processor, implements the femoral neck measuring method based on optical localization according to the first aspect.

The technical features of the above embodiments can be combined in any way. In order to simplify the description, not all the possible combinations of the technical features of the above embodiments are described. However, as long as there is no contradiction in the combinations of these technical features, they should be considered as falling within the scope recorded in this specification.

The term “comprising”, “including” or any other variation thereof herein are intended to cover non-exclusive inclusion, which includes not only those listed elements, but also other elements not explicitly listed.

The above are only some embodiments of the disclosure, but the protection scope of the disclosure is not limited to this. Any changes or substitutions within the technical scope of the disclosure are readily conceivable to a person of ordinary skills in the art, and all the changes or substitutions should be covered by the protection scope of the disclosure. Therefore, the protection scope of the disclosure shall be subjected to the protection scope of the claims.

1. A femoral neck measuring method based on optical localization, comprising:
measuring an affected limb after a femoral stem prosthesis is installed through an optical localization probe, and calculating an actual length of the femoral neck needed by the affected limb according to an osteotomy surface of the affected limb determined after the femoral stem prosthesis is installed;
determining a femoral ball prosthesis and a femoral neck prosthesis to be selected according to the calculated actual length of the femoral neck and a length of a femoral neck of a healthy limb measured before surgery; and
measuring a length of the affected limb after the femoral ball prosthesis and the femoral neck prosthesis are implanted, and restraining a length difference between the healthy limb and the affected limb by adjusting a length of the femoral neck of the affected limb.

2. The femoral neck measuring method based on optical localization of claim 1, wherein, measuring an affected limb after a femoral stem prosthesis is installed through an optical localization probe, and calculating an actual length of the femoral neck needed by the affected limb according to an osteotomy surface of the affected limb determined after the femoral stem prosthesis is installed, comprise:
measuring three non-collinear reference points on the osteotomy surface of the affected limb by using the optical localization probe, and determining coordinates of the three reference points;
pre-installing the femoral ball prosthesis and the femoral neck prosthesis selected before surgery, measuring a measuring point of the femoral ball prosthesis far away from the osteotomy surface by using the optical localization probe, and determining coordinates of the measuring point of the femoral ball prosthesis; and
determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis.

3. The femoral neck measuring method based on optical localization of claim 2, wherein determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis, comprise:
substituting coordinate variables of the three reference points into a plane formula of the osteotomy surface of the affected limb for solution;
the plane formula of the osteotomy surface of the affected limb being:
ax+by+cz+d=0;
wherein, a, b, c and d are all constants, while x, y and z respectively represent the coordinate values of the points on the osteotomy surface of the affected limb;
calculating the distance between the osteotomy surface of the affected limb and the measuring point by using a distance calculation formula;
the distance calculation formula being:
$$e = \frac{{ax}_{4} + {by}_{4} + {cz}_{4} + d}{\sqrt{{a}^{2} + {b}^{2} + {c}^{2}}} ;$$
wherein, x₄, y₄ and z₄ respectively represent the three coordinate values of the measuring point, and e represents the distance between the osteotomy surface of the affected limb and the measuring point; and
subtracting a radius length of the pre-selected femoral ball prosthesis from the distance between the osteotomy surface of the affected limb and the measuring point to obtain the actual length of the femoral neck.

4. The femoral neck measuring method based on optical localization of claim 1, wherein determining a femoral ball prosthesis and a femoral neck prosthesis to be selected according to the calculated actual length of the femoral neck and a length of a femoral neck of a healthy limb measured before surgery comprises:
selecting a femoral neck prosthesis again according to the actual length of the femoral neck and the length of the femoral neck of the healthy limb; and
determining a size of the femoral ball prosthesis according to a size of the femoral neck prosthesis selected and the length of the femoral neck of the healthy limb, and selecting a final femoral ball prosthesis.

5. The femoral neck measuring method based on optical localization of claim 4, wherein selecting a final femoral ball prosthesis comprises:
inputting the size of the femoral neck prosthesis selected into a preset femoral ball prosthesis database, and selecting the femoral ball prosthesis from femoral ball prosthesis models output from the femoral ball prosthesis database.

6. The femoral neck measuring method based on optical localization of claim 1, wherein measuring a length of the affected limb after the femoral ball prosthesis and the femoral neck prosthesis are implanted, and restraining a length difference between the healthy limb and the affected limb by adjusting a length of the femoral neck of the affected limb, comprise:
after implanting the femoral ball prosthesis and the femoral neck prosthesis into the affected limb, measuring a length between an anterior superior iliac spine and a pelma of the affected limb to obtain the length of the affected limb;
comparing the length of the affected limb with the length of the healthy limb measured before surgery, and determining the length difference between the healthy limb and the affected limb; and
judging whether the length difference between the affected limb and the healthy limb exceeds a set value for unequal lengths of both lower limbs, and in response to the length difference exceeding the set value, replacing the femoral ball prosthesis and/or the femoral neck prosthesis so that the difference between the length of the affected limb and the length of the healthy limb is within the set value for unequal lengths of both lower limbs.

7. The femoral neck measuring method based on optical localization of claim 1, wherein the set value for unequal lengths of both lower limbs is 10 mm.

8. A computer device, comprising:
a memory storing a computer program thereon; and
a processor which, when executing the computer program, implements a femoral neck measuring method based on optical localization comprising:
measuring an affected limb after a femoral stem prosthesis is installed through an optical localization probe, and calculating an actual length of the femoral neck needed by the affected limb according to an osteotomy surface of the affected limb determined after the femoral stem prosthesis is installed;
determining a femoral ball prosthesis and a femoral neck prosthesis to be selected according to the calculated actual length of the femoral neck and a length of a femoral neck of a healthy limb measured before surgery; and
measuring a length of the affected limb after the femoral ball prosthesis and the femoral neck prosthesis are implanted, and restraining a length difference between the healthy limb and the affected limb by adjusting a length of the femoral neck of the affected limb.

9. The computer device of claim 8, wherein, measuring an affected limb after a femoral stem prosthesis is installed through an optical localization probe, and calculating an actual length of the femoral neck needed by the affected limb according to an osteotomy surface of the affected limb determined after the femoral stem prosthesis is installed, comprise:
measuring three non-collinear reference points on the osteotomy surface of the affected limb by using the optical localization probe, and determining coordinates of the three reference points;
pre-installing the femoral ball prosthesis and the femoral neck prosthesis selected before surgery, measuring a measuring point of the femoral ball prosthesis far away from the osteotomy surface by using the optical localization probe, and determining coordinates of the measuring point of the femoral ball prosthesis; and
determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis.

10. The computer device of claim 9, wherein determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis, comprise:
substituting coordinate variables of the three reference points into a plane formula of the osteotomy surface of the affected limb for solution;
the plane formula of the osteotomy surface of the affected limb being:
ax+by+cz+d=0;
wherein, a, b, c and d are all constants, while x, y and z respectively represent the coordinate values of the points on the osteotomy surface of the affected limb;
calculating the distance between the osteotomy surface of the affected limb and the measuring point by using a distance calculation formula;
the distance calculation formula being:
$$e = \frac{{ax}_{4} + {by}_{4} + {cz}_{4} + d}{\sqrt{{a}^{2} + {b}^{2} + {c}^{2}}} ;$$
wherein, x₄, y₄ and z₄ respectively represent the three coordinate values of the measuring point, and e represents the distance between the osteotomy surface of the affected limb and the measuring point; and
subtracting a radius length of the pre-selected femoral ball prosthesis from the distance between the osteotomy surface of the affected limb and the measuring point to obtain the actual length of the femoral neck.

11. The computer device of claim 8, wherein determining a femoral ball prosthesis and a femoral neck prosthesis to be selected according to the calculated actual length of the femoral neck and a length of a femoral neck of a healthy limb measured before surgery comprises:
selecting a femoral neck prosthesis again according to the actual length of the femoral neck and the length of the femoral neck of the healthy limb; and
determining a size of the femoral ball prosthesis according to a size of the femoral neck prosthesis selected and the length of the femoral neck of the healthy limb, and selecting a final femoral ball prosthesis.

12. The computer device of claim 11, wherein selecting a final femoral ball prosthesis comprises:
inputting the size of the femoral neck prosthesis selected into a preset femoral ball prosthesis database, and selecting the femoral ball prosthesis from femoral ball prosthesis models output from the femoral ball prosthesis database.

13. The computer device of claim 8, wherein measuring a length of the affected limb after the femoral ball prosthesis and the femoral neck prosthesis are implanted, and restraining a length difference between the healthy limb and the affected limb by adjusting a length of the femoral neck of the affected limb, comprise:
after implanting the femoral ball prosthesis and the femoral neck prosthesis into the affected limb, measuring a length between an anterior superior iliac spine and a pelma of the affected limb to obtain the length of the affected limb;
comparing the length of the affected limb with the length of the healthy limb measured before surgery, and determining the length difference between the healthy limb and the affected limb; and
judging whether the length difference between the affected limb and the healthy limb exceeds a set value for unequal lengths of both lower limbs, and in response to the length difference exceeding the set value, replacing the femoral ball prosthesis and/or the femoral neck prosthesis so that the difference between the length of the affected limb and the length of the healthy limb is within the set value for unequal lengths of both lower limbs.

14. The computer device of claim 8, wherein the set value for unequal lengths of both lower limbs is 10 mm.

15. A non-transitory computer readable storage medium storing a computer program thereon which, when executed by a processor, implements a femoral neck measuring method based on optical localization comprising:
measuring an affected limb after a femoral stem prosthesis is installed through an optical localization probe, and calculating an actual length of the femoral neck needed by the affected limb according to an osteotomy surface of the affected limb determined after the femoral stem prosthesis is installed;
determining a femoral ball prosthesis and a femoral neck prosthesis to be selected according to the calculated actual length of the femoral neck and a length of a femoral neck of a healthy limb measured before surgery; and
measuring a length of the affected limb after the femoral ball prosthesis and the femoral neck prosthesis are implanted, and restraining a length difference between the healthy limb and the affected limb by adjusting a length of the femoral neck of the affected limb.

16. The non-transitory computer readable storage medium of claim 15, wherein, measuring an affected limb after a femoral stem prosthesis is installed through an optical localization probe, and calculating an actual length of the femoral neck needed by the affected limb according to an osteotomy surface of the affected limb determined after the femoral stem prosthesis is installed, comprise:
measuring three non-collinear reference points on the osteotomy surface of the affected limb by using the optical localization probe, and determining coordinates of the three reference points;
pre-installing the femoral ball prosthesis and the femoral neck prosthesis selected before surgery, measuring a measuring point of the femoral ball prosthesis far away from the osteotomy surface by using the optical localization probe, and determining coordinates of the measuring point of the femoral ball prosthesis; and
determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis.

17. The non-transitory computer readable storage medium of claim 16, wherein determining a plane position of the osteotomy surface of the affected limb according to the coordinates of the three reference points, calculating a distance between the osteotomy surface of the affected limb and the measuring point of the femoral ball prosthesis, and determining the actual length of the femoral neck with reference to a size of the femoral neck prosthesis, comprise:
substituting coordinate variables of the three reference points into a plane formula of the osteotomy surface of the affected limb for solution;
the plane formula of the osteotomy surface of the affected limb being:
ax+by+cz+d=0;
wherein, a, b, c and d are all constants, while x, y and z respectively represent the coordinate values of the points on the osteotomy surface of the affected limb;
calculating the distance between the osteotomy surface of the affected limb and the measuring point by using a distance calculation formula;
the distance calculation formula being:
$$e = \frac{{ax}_{4} + {by}_{4} + {cz}_{4} + d}{\sqrt{{a}^{2} + {b}^{2} + {c}^{2}}} ;$$
wherein, x₄, y₄ and z₄ respectively represent the three coordinate values of the measuring point, and e represents the distance between the osteotomy surface of the affected limb and the measuring point; and
subtracting a radius length of the pre-selected femoral ball prosthesis from the distance between the osteotomy surface of the affected limb and the measuring point to obtain the actual length of the femoral neck.

18. The non-transitory computer readable storage medium of claim 15, wherein determining a femoral ball prosthesis and a femoral neck prosthesis to be selected according to the calculated actual length of the femoral neck and a length of a femoral neck of a healthy limb measured before surgery comprises:
selecting a femoral neck prosthesis again according to the actual length of the femoral neck and the length of the femoral neck of the healthy limb; and
determining a size of the femoral ball prosthesis according to a size of the femoral neck prosthesis selected and the length of the femoral neck of the healthy limb, and selecting a final femoral ball prosthesis.

19. The non-transitory computer readable storage medium of claim 18, wherein selecting a final femoral ball prosthesis comprises:
inputting the size of the femoral neck prosthesis selected into a preset femoral ball prosthesis database, and selecting the femoral ball prosthesis from femoral ball prosthesis models output from the femoral ball prosthesis database.

20. The non-transitory computer readable storage medium of claim 15, wherein measuring a length of the affected limb after the femoral ball prosthesis and the femoral neck prosthesis are implanted, and restraining a length difference between the healthy limb and the affected limb by adjusting a length of the femoral neck of the affected limb, comprise:
after implanting the femoral ball prosthesis and the femoral neck prosthesis into the affected limb, measuring a length between an anterior superior iliac spine and a pelma of the affected limb to obtain the length of the affected limb;
comparing the length of the affected limb with the length of the healthy limb measured before surgery, and determining the length difference between the healthy limb and the affected limb; and
judging whether the length difference between the affected limb and the healthy limb exceeds a set value for unequal lengths of both lower limbs, and in response to the length difference exceeding the set value, replacing the femoral ball prosthesis and/or the femoral neck prosthesis so that the difference between the length of the affected limb and the length of the healthy limb is within the set value for unequal lengths of both lower limbs.
